(19) United States
(12) Patent Application Publication
(10) Pub. No.: US 2026/0036572 A1
(43) Pub. Date: Feb. 5, 2026

(54) METHODS FOR MITOCHONDRIAL pH PROBES

(71) Applicant: Miphic, Inc., Wilmington, DE (US)

(72) Inventor: Gennady Nikitin, Ulset (NO)

(73) Assignee: Miphic, Inc., Wilmington, DE (US)

(21) Appl. No.: 18/788,972

(22) Filed: Jul. 30, 2024

(51) Int. Cl. G01N 33/50; G01N 21/64

(52) U.S. Cl. CPC G01N 33/5079; G01N 21/6428; G01N 2021/6439

(57) ABSTRACT

The invention relates to improved methods for using fluorescence-based probes which are optimized to have a quasilinear response in the pH range which is physiologically relevant for mitochondria to measure mitochondrial pH, various mitochondrial metabolism parameters, and drug efflux capacity. These probes can be used to measure the functional parameters of mitochondria of any cell type, based on the measurement of mitochondrial pH and its changes upon various mitochondrial metabolism stimuli and inhibitions.

### FIELD OF THE INVENTION

The present invention relates to mitochondrial probes and their uses. The probes comprise a pH-independent fluorophore linked to a pH-dependent fluorophore, and may be used alone or in conjunction with other fluorescent dyes to investigate mitochondrial function.

### BACKGROUND TO THE INVENTION

Mitochondria are rod-shaped organelles in a cell that convert nutrients into adenosine triphosphate (ATP), using oxygen as an oxidant, thereby providing energy for the cell to drive the cell's metabolic activities. This process is known as aerobic respiration and is the primary mode of producing usable energy for biological processes, such as biosynthesis, locomotion, or transportation of molecules across cell membranes in both animal and plant cells.

Without mitochondria, most animal and plant cells would only be able to obtain energy from anaerobic respiration which is around 15 times less efficient than aerobic respiration. In addition to this critical energy production function, mitochondria also regulate cellular redox states, generate most of the cellular reactive oxygen species, and initiate cellular apoptosis.

The number of mitochondria present in a cell depends upon the metabolic requirements of that cell, and may range from a single large mitochondrion to thousands of the organelles. Mitochondria are found in nearly all eukaryotes, including plants, animals, fungi, and protists, and are large enough to be observed with light microscopy.

Mitochondrial function is also known to affect or be affected by certain diseases and their treatments. Tumor cells often exhibit metabolic reprogramming involving a range of metabolic features, including aberrant mitochondrial metabolism, abnormal expression of metabolic enzymes, and increased dependence on glycolysis for ATP generation and biomolecule production. They also frequently exhibit dysregulation in other mitochondrial parameters, including deregulated mtDNA content, increased ROS production, and defects in oxidative phosphorylation, suggesting that these alterations can be indicative of carcinogenesis.

Mitochondrial metabolic pathways have a high potential for anti-cancer therapy. The practical clinical situation, however, remains very complicated. There are several types of metabolic adaptations in mitochondria of cancer cells, and an effective therapy must rely on the precise diagnosis of which type is present in a particular patient. Without such precision, targeting of mitochondria by anti-cancer therapies can have an up to 60% failure rate. Previously, there has been no reliable method to test the function of mitochondria in cancer cells derived from a clinical patient. Moreover, there is no reliable method for testing the potential drug efflux capacity for various treatments among clinical patients.

WO 2019/180105 A1 describes the synthesis of and use of mitochondrial pH probes.

WO 2022/207813 A1 describes the synthesis of and use of mitochondrial pH probes of improved biologic stability.

### SUMMARY OF THE INVENTION

In a first aspect, the invention relates to improved methods for using fluorescence-based probes which are optimized to have a quasilinear response in the pH range which is physiologically relevant for mitochondria to measure mitochondrial pH, various mitochondrial metabolism parameters, and drug efflux capacity. These probes can be used to measure the functional parameters of mitochondria of any cell type, based on the measurement of mitochondrial pH and its changes upon various mitochondrial metabolism stimuli and inhibitions.

In a second aspect, the invention relates to methods for using fluorescence-based mitochondrial pH probes in conjunction with other fluorescent probes to measure mitochondrial metabolism and drug efflux capacity.

In a third aspect, the invention relates to methods for measuring the effect of a bioactive substance on mitochondrial pH, mitochondrial metabolism, or drug efflux capacity.

In a fourth aspect, the invention relates to methods of diagnosis and improving therapeutic efficacy in conditions that affect or are affected by mitochondrial function.

### DETAILED DESCRIPTION OF THE INVENTION

Two fluorescent dyes have been found to be of particular utility for use in mitochondrial probes to analyze mitochondrial metabolism. These compounds have the following structures and are referred to as 6AF-NDS-Cy3-DA and 6AF-NDS-Cy5-DA, respectively.

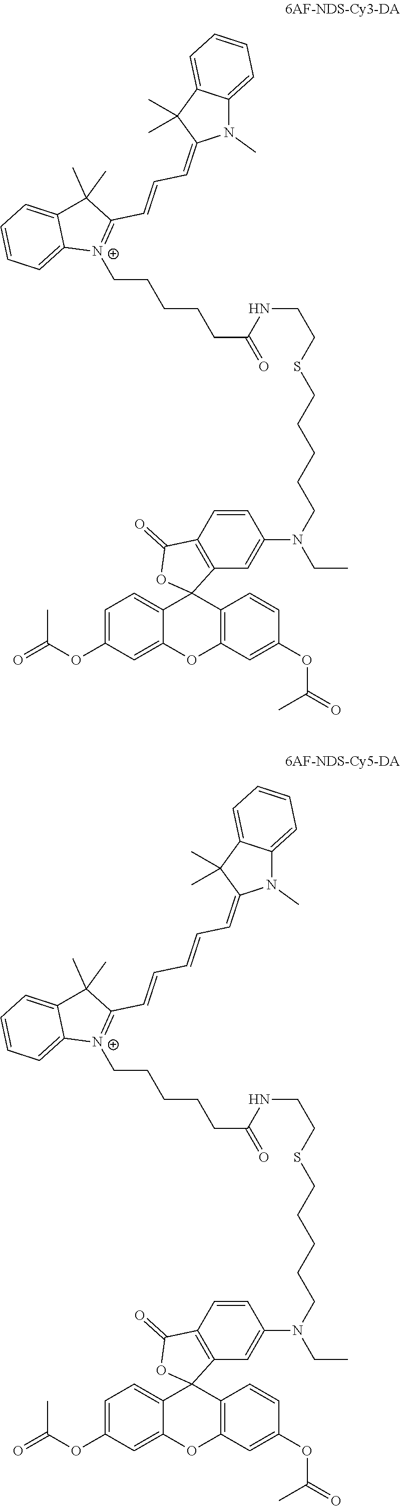 [SMILES: CCN(CCCCCSCCNC(=O)CCCCC[N+]1=C(/C=C/C=C2/N(C)c3ccccc3C2(C)C)C(C)(C)c2ccccc21)c1ccc2c(c1)C1(OC2=O)c2ccc(OC(C)=O)cc2Oc2cc(OC(C)=O)ccc21 ; CCN(CCCCCSCCNC(=O)CCCCC[N+]1=C(/C=C/C=C/C=C2/N(C)c3ccccc3C2(C)C)C(C)(C)c2ccccc21)c1ccc2c(c1)C1(OC2=O)c2ccc(OC(C)=O)cc2Oc2cc(OC(C)=O)ccc21]

6AF-NDS-Cy3-DA comprises the fluorophore 6AF (6-aminofluorescein) linked to the fluorophore Cy3, joined via a double alkyl-N-substituted linker. 6AF-NDS-Cy5-DA comprises the fluorophore 6AF (6-aminofluorescein) linked to the fluorophore Cy5, joined via a double alkyl-N-substituted linker. In each compound, the 6AF unit has been diacetylated, leading to the designation DA. The double alkyl-N-substituted linker is —(CH2)n-(C═O)—NH—CH₂CH2-S—(CH2)m-, wherein n and m are 2-8, preferably 4-6, and more preferably n and m are 5. The synthesis of such probes is described in WO 2022/207813 (PCT7EP2022/058616) to Nikitin et al. (filed 31 Mar. 2022).

The compounds of the invention are dual fluorophores. As used herein, the term “fluorophore” means a fluorescent chemical compound that can re-emit light upon light excitation, emitted light being of a different wavelength than excitation light. “Dual fluorophore” means a molecule that contains two or more covalently-linked fluorophores that are tethered together by a linker. Upon light excitation, each fluorophore can re-emit light. FRET (Forster resonance energy transfer) is a process, when the energy of light is used to excite one fluorophore (donor), while the emission light of different wavelength is produced by another fluorophore (acceptor), and that is not a typical emission wavelength of the donor fluorophore.

Prior research and applications have identified broad families of potential dual fluorophores and investigated their potential uses for measuring mitochondrial pH. The efficacy of such fluorophores can vary widely depending on the chemical characteristics of component moieties, linkers, and physiochemical conditions of the subject cells. Accordingly, the properties and utility of such dual fluorophores is highly variable and unpredictable.

A subset of dual fluorophore probes has been identified as surprisingly useful for its combination of qualities beneficial for the purposes of measuring mitochondrial function. The newly discovered qualities of these particularly useful probes, comprising 6-aminofluoroscein molecules linked to the cyanine dyes Cy3 or Cy5 wherein the linker is a double alkyl-N-substituted linker, enable new methods for determining mitochondrial pH, mitochondrial metabolism, and drug efflux capacity.

These two compounds are examples from a specific family of probes which have a pH-dependent fluorophore comprising 6-aminofluorescein (6AF), a pH-independent fluorophore comprising cyanine dye (Cy3 or Cy5), and a linker comprising a covalent chain designed to minimize any steric hindrance for the rotation of the two fluorophores around the linker relative to each other that may adversely affect the ability of the probe to travel through the cellular plasma membrane and mitochondrial membranes. Most preferably, the linker is —(CH₂)n-(C═O)—NH—CH₂CH₂—S—(CH₂)m- wherein n and m are 2-8, preferably 4-6, and more preferably n and m are 5. In 6AF-NDS-Cy3-DA and 6AF-NDS-Cy5-DA, the linker is —(CH₂)5—(C—O)—NH—CH₂CH₂—S—(CH₂)₅.

pH-Dependent Fluorophore

6-aminofluorescein (6AF) is an active form of a pH-dependent fluorophore. As used herein, the term “pH-dependent fluorophore” means a fluorophore whose fluorescence intensity changes depending on the pH of the environment it is in. A pH-dependent fluorophore is sensitive to the pH environment and exhibits variable fluorescence depending on pH.

The mitochondrial pH probe according to the present invention is in a protective (i.e. inactive) form; it is protected into an overall positive oxidation state. In this form, the probe is efficiently transported into the mitochondria of the cell. The protection is removed inside mitochondrial matrix (e.g. via an esterase) to convert the probe into an active form with a variable oxidation state so that the probe is sensitive to changes to pH.

“Inactive form” or “proactive form” means the molecule has been modified such that it exists in a positive oxidation state and that pH-dependent fluorophore exhibits no FRET to pH-independent fluorophore. “Active form” means that the molecule is configured to produce its desired effect, e.g. measure mitochondrial pH. In active form, the molecule will fluoresce in a pH-dependent manner to enable mitochondrial pH measurement. In addition, the FRET between pH-dependent and pH-independent fluorophores is activated only in active form. The molecule may be converted into an active form via removal of the protecting group(s).

“Oxidation state” means the overall charge of an atom, part of a molecule or a molecule. A single positive charge in a molecule corresponds to an oxidation state of +1 and a single negative charge corresponds to an oxidation state of −1. The oxidation state of a molecule or part of a molecule can be condition dependent or independent. For example, a nitrogen with four alkyl bonds exists in a +1 oxidation state irrespective of the surrounding conditions (e.g. pH). By comparison, an alcohol or phenol molecule ROH exemplifies a variable oxidation state as it can exist at multiple oxidation states depending on the pH of its surroundings. At low pH, the oxygen will bond to H⁺, leaving the alcohol ROH+ in a 0 oxidation state. Alternatively, at a high pH, H⁺ may be lost, leading to a molecule in a −1 oxidation state as RO⁻. As a further example, a molecule with the fixed oxidation state nitrogen and the variable oxidation state alcohol both present would display a variable oxidation state, moving from +1 in inactive form of the molecule to 0 for the active form of the molecule at low pH to −1 for the active form of the molecule at high pH.

In an inactive form, the mitochondrial pH probes of the present invention are protected such that they exist in a positive oxidation state. In a preferred embodiment, the probe is in a fixed oxidation state of +1.

By fixing the probe into a positive oxidation state, the probe will preferentially accumulate within the mitochondria. Additionally, in the inactive form the probe is much more lipophilic, thus permeating the cellular membranes much more rapidly. Mitochondria are distinguished from other cellular organelles by their inner mitochondrial membranes having a high electrochemical potential. This electrostatic potential pulls the positively-charged probe into the mitochondria leading to a concentration of probe in the mitochondria several orders of magnitude higher than the rest of the cell. The positive charge on the probe also helps with the initial delivery of the probe into the cell since the cell plasma membrane also has an electrochemical potential of the same polarity as the mitochondria, albeit at a much lower magnitude.

Once in the cytoplasm, the probe is attracted into the mitochondria, again due to its positive oxidation state. The probe then accumulates inside the mitochondria in the mitochondrial matrix. The probe is converted via cellular enzymes (for example esterases) into active form. While this conversion will take place within the whole cell, the preferential accumulation into the mitochondria due to the positive probe charge leads to an overall accumulation and entrapment inside the mitochondria.

In an inactive form, the probe is protected by one or more protecting groups (PGs). As used herein, the term “protecting group” means a group which has been introduced into a molecule by modification of a functional group which prevents said functional group from undergoing further changes (e.g. reactions) until the protecting group has been removed. Suitable protecting groups can be deprotected intracellularly.

In particular, the pH-dependent fluorophore 6AF comprises oxygen groups which can be protected to lock or cage the molecule into a positive oxidation state, and to remove FRET between the 6AF and Cy3 or Cy5. In such an embodiment, the pH-dependent fluorophore 6AF in inactive form comprises one or more protected oxygen(s). In the compounds shown here, the 6AF unit has been diacetylated to form two acetyl protecting groups on hydroxyl groups of 6-aminofluorescin.

Suitable protecting groups include alcohol protecting groups, amine protecting groups, carboxylic acid protecting groups. Examples of alcohol protecting groups include esters, formed with saturated and aromatic carboxylic acids, organic carbonate esters. Examples of amine protecting groups include amides, formed with saturated and aromatic carboxylic acids, carbamates, thiocarbamates. Examples of carboxylic acid protecting groups include acetoxymethyl ester, anhydrides, formed with saturated and aromatic carboxylic acids. Other examples of possible protecting groups include acyl, propionyl, butyryl, isobutyryl, pivaloyl or benzoyl. An especially preferred protecting group is acyl.

pH-Independent Fluorophore

Cyanine dyes (Cy3 and Cy5) are pH-independent fluorophores. “pH-independent fluorophore” means a fluorophore which emits light at a specific wavelength largely irrespective of the pH of the environment it is in. A pH-independent fluorophore therefore exhibits consistent fluorescence over variable pH conditions, for example both low and high pH.

Cy3 or Cy5 have been found to be particularly useful pH-independent fluorophores as they are cationic and lipophilic. By using a cationic and/or lipophilic fluorophore, the probe can more easily pass through the plasma and mitochondrial membranes and the positive charge allows the probe to target the mitochondria. Here, Cy3 and Cy5 have been discovered to be useful mitochondrial-targeting moieties.

### Linker

6AF-NDS-Cy3-DA and 6AF-NDS-Cy5-DA each have a linker comprising —(CH2)5-(C═O)—NH—CH2CH2-S—(CH2)5—. The linker is a covalent chain designed to minimize any steric hindrance for the rotation of the two fluorophores around the linker relative to each other that may adversely affect the ability of the probe to travel through the cellular plasma membrane and mitochondrial membranes. Most preferably, the linker is —(CH₂)n-(C═O)—NH—CH₂CH₂—S—(CH₂)m wherein n and m are 2-8, preferably 4-6, and more preferably n and m are 5. In 6AF-NDS-Cy3-DA and 6AF-NDS-Cy5-DA, the linker is —(CH2)5—(C═O)—NH-CH2CH2-S—(CH2)5.

The term “tautomer” as used herein refers to structural isomers which readily interconvert by way of a chemical reaction which may involve the migration of a proton accompanied by a switch of a single bond and adjacent double bond. Dependent on the conditions, the compounds may predominantly exist either in the keto or enol form and the invention is not intended to be limited to the particular form shown in any of the structural formulae given herein. Tautomerism of fluorescein in variable pH is known to affect fluorescent emission, such that tautomerism is a contributing factor to making aminofluorescein a pH-dependent fluorophore. Crucially, the ratio of fluorescent and non-fluorescent tautomeric forms of fluorescein is for the most part dictated by the pH of environment, which enables its use as a pH-dependent fluorophore. Accordingly, the dual fluorophore compounds of the presently invention are contemplated to include all possible tautomers in variable pH conditions.

The compounds herein described contain one or more chiral centers and may therefore exist in different stereoisomeric forms. The term “stereoisomer” refers to compounds which have identical chemical constitution but which differ in respect of the spatial arrangement of the atoms or groups. Examples of stereoisomers are enantiomers and diastereomers. The term “enantiomers” refers to two stereoisomers of a compound which are non-superimposable mirror images of one another. The term “diastereoisomers” refers to stereoisomers with two or more chiral centers which are not mirror images of one another. The invention is considered to extend to the use of diastereomers and enantiomers, as well as racemic mixtures. The compounds herein described contain one or more resonance structures where delocalized electrons contribute to resonance or mesomerism. As such, the compounds considered to include all resonance implied by the structural diagrams.

The compounds of the invention must be cationic for targeting of mitochondria; therefore they may be provided as salts. The term “pharmaceutically-acceptable salt” as used herein refers to any pharmaceutically-acceptable organic or inorganic salt of any of the compounds herein described. A pharmaceutically acceptable salt may include one or more additional molecules such as counter-ions. The counter-ions may be any organic or inorganic group which stabilizes the charge on the parent compound. If the compound for use in the invention is a base, a suitable pharmaceutically acceptable salt may be prepared by reaction of the free base with an organic or inorganic acid. If the compound for use in the invention is an acid, a suitable pharmaceutically acceptable salt may be prepared by reaction of the free acid with an organic or inorganic base. Non-limiting examples of suitable salts are described herein. The term “pharmaceutically-acceptable” means that the compound or composition is chemically and/or toxicologically compatible with other components of the formulation or with the patient's mitochondria. Preferably, the salt is a chloride, tetrafluoroborate or perchlorate, or any other strong acid's anion.

WO 2019/180105 A1 describes the synthesis of and use of mitochondrial pH probes.

WO 2022/207813 A1 describes the synthesis of and use of mitochondrial pH probes of improved biologic stability, including 6AF-NDS-Cy3-DA. Synthesis of 6AF-NDS-Cy5-DA may be achieved by similar means by replacing Cy-3-COOH with Cy5-COOH during the synthesis of 6AF-NDS-Cy3-DA.

The methods of the present invention provide advantages over methods described in the prior art for using dual fluorophore probes to measure mitochondrial pH. The probes of the present invention have been found to be particularly useful for analysis of mitochondrial metabolism.

The dual fluorophore probes of the present invention have a high sensitivity to pH and other characteristics that enable mitochondrial analysis with accuracy and reproducibility that was previously impossible. Their high sensitivity to pH also allows for new methods for accurate analysis of mitochondrial metabolism, which are described herein. Additional methods of mitochondrial analysis using complementary probes are also described herein.

### Methods of Measuring Mitochondrial pH

Previously described methods could not reliably align the fluorescence readings to a specific pH value. Due to disadvantages in the molecular structures of the various mitochondrial probes, such as lack of ratiometric readout and not sufficiently selective mitochondrial targeting, and the lack of tight pH control of the medium during their application, results could not be reliably replicated and thus the practical utility of such probes was limited.

The dual fluorophores of the present invention reliably fluoresce at set wavelengths, approximately 570 nm (fluorescein to Cy3 FRET, and Cy3) and 670 nanometers (fluorescein to Cy5 FRET, and Cy5), in response to excitation at approximately 488 nm (fluorescein to Cy3 FRET) and 550 nm (Cy3), and 488 nm (fluorescein to Cy5 FRET) and 640 nm (Cy5). Cy3 fluoresces greenish yellow (˜550 nm excitation, ˜570 nm emission), while Cy5 is fluorescent in the far-red region (˜640 excitation, 670 nm emission). Thus, the ratiometric fluorescent signal of the present invention can be used to quantify mitochondrial pH levels reliably and repeatably against known values, if the methods here described are employed.

The described methods are for analyzing cell samples in vitro. A cell sample may be collected from a patient or specimen or provided to a person of ordinary skill in the art for analysis.

In a first aspect, the methods may be used to measure the mitochondrial pH of cells in a cell sample, the method comprising the steps of:
(a) washing a cell sample with a bicarbonate-free cell culture medium with a tightly controlled pH value;
(b) contacting the cell sample with 6AF-NDS-Cy3-DA or 6AF-NDS-Cy5-DA, said compound comprising a pH-independent fluorophore linked to a pH-dependent fluorophore;
(c) rinsing the cell sample with a bicarbonate-free cell culture medium with a tightly controlled pH value;
(d) measuring a ratio of fluorescence between the pH-independent fluorophore and the pH-dependent fluorophore, where the value of background fluorescence was subtracted, to increase signal-to-noise ratio; and
(e) determining the mitochondrial pH using said ratio of fluorescence.

This method comprises first washing a cell sample with a bicarbonate-free cell culture medium with a tightly-controlled pH value. Here, “tightly-controlled pH value” means the bicarbonate-free cell culture medium has a pH value that has a pH variance that is no greater than 0.02 pH unit. Preferably, the pH variance is no greater than 0.01 pH unit. For example, a bicarbonate-free cell culture medium of physiological pH 7.4 would have a tightly-controlled pH value if it maintained a pH of between 7.38 and 7.42, inclusive. Preferably, a bicarbonate-free cell culture medium of physiological pH 7.4 would have a tightly-controlled pH value if it maintained a pH of between 7.39 and 7.41.

In some embodiments, the bicarbonate-free cell culture medium is tightly-controlled to physiological pH of 7.4±0.02, and preferably to 7.40±0.01. Here, “physiological pH” means the pH of the human body in the absence of pathological states, which is between 7.25 and 7.45, with the average at 7.40.

In some embodiments, the bicarbonate-free cell culture medium is tightly-controlled to non-physiological pH to reflect pathological states. In hypoxic conditions, low oxygen levels can increase anaerobic metabolic pathways and increase lactic acid, lowering pH. Metabolic or hypercapnic acidosis may also be caused by disease or conditions related to the treatment of disease. Rapid growth of malignant cancer cells may also increase hypoxia, reducing the clearance of acidifying waste products. Accordingly, the target pH may be tightly controlled to tumor-mimicking or hypoxic pH levels below the physiological pH 7.4, such as 7.2, 7.0, or 6.8. In other conditions, such as where intracellular tumor milieu is slightly or strongly alkaline or the patient is suffering from metabolic alkalosis, the target pH may be tightly controlled to pH levels above the physiological pH 7.4, such as 7.5, 7.6, or 7.8. In all embodiments, the pH level of the bicarbonate-free cell culture medium used in the washing step is tightly-controlled.

Here, “washing” the cell sample means saturating, inundating, or otherwise contacting the cell sample with the bicarbonate-free cell culture medium in ways known to those of skill in the art of cell sample analysis or as directed by physicians, pathologists, or medical researchers. Different cell samples may demand different amounts of bicarbonate-free cell culture medium, residence times, or washing techniques. Washing the cell sample with a bicarbonate-free cell culture medium which has a tightly-controlled pH level allows the extracellular medium of the cell sample to achieve a target pH approaching the tightly-controlled pH of the cell culture medium.

The method to measure mitochondrial pH comprises next contacting the cell sample with 6AF-NDS-Cy3-DA and/or 6AF-NDS-Cy5-DA, said compounds comprising a pH-independent fluorophore linked to a pH-dependent fluorophore. As noted above, the properties of these compounds make them especially useful for passing from the extracellular medium of the cell sample into the cytoplasm of the cells, and from there into the mitochondria. Once they have entered the mitochondria, the protecting groups of the pH dependent fluorophore are removed, converting the compounds from an inactive to an active form. The conditions of the cell sample and the types of cells being analyzed can vary, which means that uptake rates into the cytoplasm and mitochondria may also vary. Accordingly, the contact time and concentration may be adjusted in ways known to those of skill in the art of cell sample analysis or as directed by physicians, pathologists, or medical researchers.

The method to measure mitochondrial pH next comprises rinsing the cell sample with a bicarbonate-free cell culture medium. In some embodiments, the bicarbonate-free cell culture medium for rinsing also has a tightly-controlled pH value. Here, “rinsing” means contacting the cell sample with the bicarbonate-free cell culture medium in ways known to those of skill in the art of cell sample analysis or as directed by physicians, pathologists, or medical researchers. Rinsing the cell sample can remove excess fluorophore probe from the extracellular medium that has not been absorbed by the cells. As the last step after rinsing, cell sample is supplied with the medium with a tightly-controlled pH value in which the actual measurements are subsequently performed.

The method to measure mitochondrial pH further comprises measuring a ratio of fluorescence between the pH-independent fluorophore and the pH-dependent fluorophore. Measuring the ratio of fluorescence intensities approach avoids fluorophore concentration effects from influencing the pH measurement. If a probe comprises a single pH dependent fluorophore, the intensity of the emission will be dependent not only on pH but also on the fluorophore concentration. This makes it difficult to compare results between different biological samples. By applying a ratiometric approach, internal normalization of the fluorescence readings is possible. While the absolute fluorescence intensities between different samples may vary depending on the fluorophore concentrations, the fluorescence ratio will remain independent of concentration and will only vary depending on pH.

With the ratiometric probes of the present invention, while the absolute fluorescent intensities of different samples may vary dependent on the corresponding fluorophore concentrations, the fluorescence ratio remains independent of the concentration and only responds to the change in pH. The two fluorophores have spectrally separated fluorescence bands and/or absorption bands. This improves the sensitivity of the readings and allows for more accurate measurements to be taken.

When two emission or excitation spectral bands used for the ratiometric approach correspond to the same single fluorophore, these bands are usually highly overlapping spectrally. This makes the pH-dependent fluorescence ratio change relatively small and thus the probe sensitivity low. While it is possible to use specially designed optical filters to increase probe sensitivity, this requires modification of the microscopes and thus causes impracticality. In contrast, if the two emission/excitation bands are completely spectrally separated, and are within the standard optical filter wavelength range, then the sensitivity of the probe is greatly increased and can be used with any standard imaging system. Here, 6-aminofluorescin is excited in the blue range, while Cy3 and Cy5 are excited in the green or red ranges, respectively, allowing the fluorescence corresponding to the excitation of each fluorophore to be measured with high sensitivity.

In a preferred embodiment, the ratiometric analysis compares the emission wavelength of the pH-independent fluorophore (Cy3 or Cy5) under two excitation states. When the pH-dependent fluorophore (fluorescin, i.e., 6AF) is excited at its characteristic wavelength, the attached pH-independent fluorophore (Cy3 or Cy5) fluoresces because of FRET. This fluorescence is measured and compared to the fluorescence of the pH-independent fluorophore (Cy3 or Cy5) when excited at its own characteristic wavelength. Accordingly, “a ratio of fluorescence between the pH-independent fluorophore and the pH-dependent fluorophore” includes measuring the emission wavelength of a single fluorophore (Cy3 or Cy5) under distinct excitation wavelengths which variably excite the pH-dependent (6AF) and pH-independent (Cy3 or Cy5) fluorophores, as the excitation of the pH-dependent fluorophore (6AF) causes emission from the pH-independent fluorophore (Cy3 or Cy5) because of FRET.

Collecting fluorescence values for ratiometric comparison may be achieved by methods known to those skilled in the art of cell sample analysis. Known methods of spectroscopy, microscopy, and computer analysis may be employed to obtain the absolute intensities and wavelengths of emissions from the cell sample when excited by known methods of excitation sources. The excitation source or measured emission may be reflected, lensed, filtered, detected, amplified, or visualized using devices and methods known to those of skill in the art. Preferably, the value of background fluorescence is subtracted to increase signal-to-noise ratio.

Plate readers can provide more detailed data with the higher screening speed than other methods. In addition, they often come in the format of high-throughput screening (HTS) imagers, which allow to work with image arrays and extract much more useful data from the measurements, such as of mitochondrial functions on a single-cell level; and increase the sensitivity of the signal by subtracting the background fluorescence values. Other methods, including but not limited plate-reading bulk analysis, can be used to analyze the stained cells. Overall, the staining and washing procedures can be performed within 30 minutes, after which the cells are ready for analysis.

The method to measure mitochondrial pH comprises the final step of using the ratiometric measurement of fluorescence from the pH-dependent fluorophore and the pH-independent fluorophore to determine the pH of the mitochondria. To do so, the measured ratiometric measurement may be compared to known emission ratios of the dual fluorophore compounds prepared by those of skill in the art. For example, the dual fluorophore compounds could be activated by removing the protecting groups in tightly-controlled pH conditions across a range of known pH values. The emission values from the excitement of the pH-dependent and pH-independent fluorophores and their ratios in each pH condition could then be used to create a reference point for the emission values and their ratios measured in the cell samples. In some embodiments, the emission value ratio for the dual fluorophore probe has been repeatably measured in known and repeatable conditions in a range of pH values such that no calibration step is necessary because the experimental emission value ratios can be compared to established standard emission value ratios. Mitochondrial pH is relevant to mitochondrial metabolism because mitochondrial ATP energy production depends on proton (H⁺ ions) pumps to create a diffusion gradient across an inner mitochondrial membrane. Accordingly, the specific pH value (i.e., relative abundance of H⁺) within and around the mitochondria directly influences its biological processes and changes to mitochondrial pH can lead to mitochondrial stimulation or dysfunction. The ability to accurately and sensitively measure mitochondrial pH within living cells can provide important insights into mitochondrial biology, and the dynamics of the living cell as a whole.

Normal cells primarily produce energy through glycolysis followed by mitochondrial citric acid cycle and oxidative phosphorylation. However, most cancer cells predominantly produce their energy through a high rate of glycolysis followed by lactic acid fermentation even in the presence of abundant oxygen. “Acrobic glycolysis” is less efficient than oxidative phosphorylation in terms of adenosine triphosphate production, but leads to the increased generation of additional metabolites that may particularly benefit proliferating cells such as cancer cells.

In healthy mitochondria, ATP synthase acts in its forward mode, wherein protons flow into the mitochondrial matrix to produce ATP from ADP. In cancer cells, ATP synthase often acts in reverse mode, wherein ATP is converted to ADP and protons are pumped out of the mitochondria. The ATP synthase inhibitor oligomycin inhibits proton conductance by ATP synthase, removing its contribution to the proton gradient. If mitochondrial ATP synthase acts in the normal mode, inhibition with oligomycin would increase mitochondrial pH, while in the reverse mode it would decrease mitochondrial pH, in the absence of other compensatory mitochondrial processes. In the case of pre-existing natural inhibition of ATP synthase, inhibition with oligomycin would not noticeably change mitochondrial pH. Consequently, the measurement of mitochondrial pH in the presence and absence of an ATP synthase inhibitor may be used to determine the extent to which the ATP synthase molecules are acting in forward or reverse mode, and hence to give an indication of the cancerous nature of the cells. In yet a further embodiment, therefore, the current invention provides a method of obtaining an indication of whether a subject's mitochondria may be usable as a tumor-specific target in a subject suffering from cancer.

In some embodiments, a second stain or dye is added to the cell sample before the rinsing step. The second stain or dye can be used to augment the cell sample analysis by providing additional information relating to cell function or cell characteristics. In preferred embodiments, the second stain or dye is fluorescent, so that its fluorescent emission can be analyzed using the same equipment and at the same time as the dual fluorophore probe's fluorescent emission is analyzed.

In some embodiments, the second stain or dye comprises a dead cell marker and/or a live cell marker. In one embodiment, the second dye added before rinsing comprises 4′,6-diamidino-2-phenylindole (“DAPI”) (CAS 28718-90-3). DAPI is a useful dye for nuclear quantification and has been utilized in numerous assays, such as live or fixed cell staining, cell viability assays, flow cytometry, cell cycle analysis, mycoplasma contamination detection and in fluorescence microscopy. For example, DAPI may be used to assist counting the number of dead cell nuclei in a cell sample while the probes of the present invention can analyze the location and pH of mitochondria in those cells, allowing for an analysis of mitochondria in live cells, with simultaneous estimation of percentage of the viable cells in the sample.

In other embodiments, the second dye added before rinsing comprises a calcein, such as calcein violet and/or calcein green (CAS 48504-34-1). Calcein violet is a blue fluorescent molecule that enters the membranes of dead cells and may be used to evaluate cell membrane integrity. Calcein violet AM is modified using acetoxymethyl (AM) and can passively diffuse across the membranes of live cells. While inside the live cell, calcein violet AM is hydrolyzed by esterases into the fluorescent calcein violet which can then be measured using fluorescence microscopy, flow cytometer, or fluorescent microplate reader. It has an excitation peak at 399 nm and an emission peak at 449 nm. Calcein green has similar properties to calcein violet, but has an excitation peak at 493 nm and an emission peak at 512 nm.

### Methods of Measuring Mitochondrial Metabolism and Drug Efflux Capacity

In a second aspect, the methods can be used to estimate mitochondrial drug efflux capacity, the method comprising the steps of:
(a) washing a cell sample with a bicarbonate-free cell culture medium with a tightly controlled pH value;
(b) contacting the cell sample with 6AF-NDS-Cy3-DA or 6AF-NDS-Cy5-DA, said compound comprising a pH-independent fluorophore linked to a pH-dependent fluorophore;
(c) rinsing the cell sample with a bicarbonate-free cell culture medium;
(d) contacting the cell sample with a second fluorescent dye;
(c) measuring a ratio of fluorescence between the pH-independent fluorophore, the pH-dependent fluorophore, and the ratio of florescence between second fluorescent dye and pH-independent fluorophore of the first compound; and
(f) repeating the step (e) two or more times, and
(g) estimating mitochondrial drug efflux capacity using the change in these ratios of fluorescence over time.

In some embodiments, the second fluorescent dye comprises and/or is a total mitochondrial potential reporting agent. In a preferred embodiment, a second fluorescent dye is added to the cell sample after the rinsing step. In such an embodiment, the preferred second fluorescent dye comprises a rhodamine, particularly rhodamine 800 (CAS 137993-41-0) and/or rhodamine 700 (CAS 63561-42-2). Use of rhodamine 800 or rhodamine 700 together with the mitochondrial pH probe allows not only the analysis of mitochondrial metabolism, but also the drug efflux capacity by exploiting the influence of total mitochondrial membrane potential of mitochondria to keep the mitochondrial concentration and consequently fluorescence of rhodamine constant, while monitoring the decrease in mitochondrial concentration and consequently the fluorescence of pH-independent channel of the mitochondrial pH probe over time.

Total mitochondrial membrane potential is one of the most important parameters that control mitochondrial function, and the viability of a cell in general. This parameter is not equivalent to mitochondrial pH gradient, although this does contribute to total mitochondrial membrane potential. Mitochondrial pH gradient is directly linked to the energetic function of mitochondria-such as in ATP production, oxidative phosphorylation, and the beta-oxidation of fatty acids. Total mitochondrial membrane potential is involved in homeostasis of mitochondria, such as in keeping mitochondrial integrity and prevention of mitochondria-triggered cellular apoptosis, and in regulation of mitophagy which eliminates mitochondria that have low mitochondrial potential values. It also affects the levels of oxidative stress in mitochondria. Thus, both total mitochondrial membrane potential and mitochondrial pH are important and complementary functional parameters of mitochondria, and together reflect core aspects of mitochondrial metabolism. “Mitochondrial metabolism” here relates to the mitochondria's potential or actual ability to carry out biological processes, such as generating adenosine triphosphate (ATP) through oxidative phosphorylation, redox signaling, steroid synthesis, lipid metabolism, generating macromolecular precursors, biosynthesis of fatty acids, cholesterol, or amino acids, etc. which may be approximated by measuring a range of parameters related to regulation of mitochondrial pH and total mitochondrial potential.

Rhodamine 700 (CAS 137993-41-0) and rhodamine 800 (CAS 63561-42-2) are used as total mitochondrial membrane potential-dependent probes, they emit fluorescence in a near-infrared region-being excited by deep-red light (˜640 nm). They are spectrally compatible with 6AF-NDS-Cy3-DA mitochondrial pH probe (with emission in a red region, excitation by blue ˜488 nm and green light ˜520 nm). Ester groups are found in more commonly used total mitochondrial potential dependent dyes such as TMRM or rhodamine 6G. As rhodamine 700 and rhodamine 800 do not contain ester groups, they are more chemically stable inside of mitochondria. This is especially important because ester-containing rhodamines, such as TMRM and rhodamine 6G, are converted into neutrally charged, total mitochondrial potential-independent fluorophores by esterases, making their fluorescent readout unsuitable for the purposes of this invention. The current invention relates to a combined readout of mitochondrial pH probe and total mitochondrial membrane potential-dependent probe, using rhodamine 700 or 800, and correlate them in the context of different metabolic tests. The correlational values obtained from the current invention provide information that cannot be achieved by measuring the signal from mitochondrial pH probe or total mitochondrial potential-dependent probe alone.

When rhodamine 700 or 800 is used to analyze total mitochondrial membrane potential concurrently with the dual fluorophore dyes' use to analyze mitochondrial pH, additional ratiometric analysis can provide additional information about mitochondrial metabolism and drug efflux capacity. “Drug efflux capacity” means the ability of a cell or organelle (such as mitochondria) to remove or clear a drug or other molecule from within their membranes. Most tumors have high heterogeneity and are composed of various types of tumor cells with different characteristics. This heterogeneity can contribute to acquisition of drug resistance, because a particular cell population with resistance to anti-cancer drugs will be selected and become dominant in tumors after treatment. Anti-cancer drugs can be pumped out by highly expressed drug efflux pumps, and this can prevent drugs from working properly. Therefore, the ability to estimate drug efflux capacity in cancer cells can provide a high medical value.

Here, a ratiometric analysis of the ratio of rhodamine 700 or 800 fluorescent emissions can be made in relation to the pH-independent fluorophore (Cy3 or Cy5) of the mitochondrial pH probe, while another ratiometric analysis can be made for the ratio of fluorescence between the pH-dependent fluorophore (Cy3 or Cy5) and the pH-independent fluorophore (6AF) of the mitochondrial pH probe as described herein. Because the rhodamine is added after the rinsing step, the dual fluorophore dye (mitochondrial pH probe) will have already accumulated in the mitochondrial matrix as the rhodamine concentration is equilibrated across the inner mitochondrial membrane, in accordance to the value of total mitochondrial potential. As the two ratiometric ratios are measured over time, the rate at which the dual fluorophore dye is moved out from the mitochondria (effluxed) can be estimated as its intensity of its pH-independent fluorophore decreases relative to the reference of the rhodamine emission which stays constant, because the equilibrium between its concentrations inside and outside of the mitochondrial matrix is maintained by the total mitochondrial potential. On the contrast, the dual fluorophore mitochondrial pH probe is not retained inside mitochondria by the total mitochondrial potential, because its active form does not have a positive oxidation state.

Because drug efflux capacity of certain cells or organelles can be of critical importance to the efficacy of therapies targeting such cells or organelles, this measurement of mitochondrial drug efflux capacity can be used to assess the potential efficacy of therapies targeting mitochondria. For example, if a drug requires a long residence time in the mitochondria of tumor cells to be effective, but the mitochondria of tumor cells are found (using the described method) to have a high efflux capacity meaning that residence time for the drug in the mitochondria will be short, such a drug could be understood to have a lower potential efficacy for that tumor.

### Methods of Determining the Effect of a Bioactive Substance

In a third aspect, the methods can be used to determine the effect of a bioactive substance upon mitochondrial pH, the method comprising the steps of:
(a) washing a cell sample with a bicarbonate-free cell culture medium with a tightly controlled pH value;
(b) contacting the cell sample with 6AF-NDS-Cy3-DA and/or 6AF-NDS-Cy5-DA, said compound comprising a pH-independent fluorophore linked to a pH-dependent fluorophore;
(c) rinsing the cell sample with a bicarbonate-free cell culture medium with a tightly controlled pH value;
(d) measuring a first ratio of fluorescence between the pH-independent fluorophore and the pH-dependent fluorophore;
(c) contacting the cell sample with a bioactive substance, neutral vehicle, or leaving the sample for a period of time,
(f) measuring a second ratio of fluorescence between the pH-independent fluorophore and the pH dependent fluorophore; and
(g) determining the effect of the bioactive substance on mitochondrial pH using the first ratio of step (d) and second ratio of obtained in step (f).

The effect of the bioactive substance upon mitochondrial pH may be measured against a control using a neutral vehicle or no substance. Methods for establishing an experimental group and control group are known in the art, and may vary depending on the cell sample and bioactive substance to be tested.

In another embodiment of the invention, the analytical methods are used to determine mitochondrial pH and estimate mitochondrial metabolism, and drug efflux capacity can be employed to determine the effect of a bioactive substance upon mitochondrial function. As mitochondrial function can affect or be affected by disease pathology or as a result or side effect of treatment, understanding the effect of substances on mitochondrial function may lead to improved therapies or other medical treatment results. Here, “bioactive substance” means any element, molecule, mixture, solution, drug, enzyme, protein, etc., or combination thereof that affects the biological function of an organelle, cell, tissue, or organism.

For methods of determining the effect of a bioactive substance on mitochondrial pH, the previously described methods for determining mitochondrial pH are performed using the dual fluorophore dyes described herein. After the initial ratiometric measurement of emissions from pH-dependent fluorophore excitement against emissions from pH-independent fluorophore excitement in the cell sample is completed, the cell sample is contacted with a bioactive substance that is to be assessed for its effect on mitochondrial pH. The concentration and duration of contact will depend on the bioactive substance, the nature of the cell sample, and the experimental parameters understood by those of skill of the art of cell sample analysis or as directed by physicians, pathologists, or medical researchers. It is also possible to contact the bioactive substance with the cell sample at an earlier stage if the bioactive substance, cell sample, or experimental conditions make doing so necessary or advantageous.

After an initial ratiometric measurement of emissions from pH-dependent fluorophore excitement against emissions from pH-independent fluorophore excitement in the cell sample, subsequent ratiometric measurements are then made to determine the change in emission ratios over time in the presence of the bioactive substance. Accordingly, the effect of the bioactive substance on mitochondrial pH can be determined by measuring and analyzing the change in ratiometric measurements. Even though a bioactive substance may occlude some emission or change other factors in the cell sample that determine the total fluorescent output of the dual fluorophores, the dual fluorophore dyes remain in a constant ratio of one pH-independent fluorophore to one pH-dependent fluorophore such that the ratio of their fluorescence can be used to reliably determine mitochondrial pH.

In some embodiments, the methods for determining the effect of a bioactive substance on mitochondrial pH will include contacting the cell sample with a dead cell marker and/or live cell marker before rinsing. In such embodiments, the dead cell marker and/or live cell marker comprises and/or is 4′,6-diamidino-2-phenylindole (DAPI) (CAS 28718-90-3), calcein violet, calcein violet AM, and/or calcein green (CAS 48504-34-1), calcein green AM.

In a fourth aspect, the methods can be used to determine the effect of a bioactive substance upon mitochondrial metabolism or mitochondrial drug efflux capacity, the method comprising the steps of:
(a) washing a cell sample with a bicarbonate-free cell culture medium with a tightly controlled pH value;
(b) contacting the cell sample with 6AF-NDS-Cy3-DA or 6AF-NDS-Cy5-DA, said compound comprising a pH-independent fluorophore linked to a pH-dependent fluorophore;
(c) rinsing the cell sample with a bicarbonate-free cell culture medium with a tightly controlled pH value;
(d) contacting the cell sample with a second fluorescent dye;
(e) measuring a first ratio of fluorescence between the pH-independent fluorophore, and the second fluorescent dye;
(f) contacting the cell sample with a bioactive substance, neutral vehicle, or leaving the sample for a period of time; and
(g) measuring a second ratio of fluorescence between the pH-independent fluorophore, and the second fluorescent dye;
(h) determining the effect of the bioactive substance upon mitochondrial metabolism and/or drug efflux capacity using the first ratio of step (e) and second ratio obtained in step (g), as compared to the results of similar measurements obtained without the step (f), i.e. in the absence of a bioactive substance.

For methods of determining the effect of a bioactive substance on mitochondrial metabolism and drug efflux capacity, the previously described methods for determining mitochondrial metabolism and drug efflux capacity are performed using the dual fluorophore dyes and a second dye as described herein. After the initial ratiometric measurements of emissions from pH-independent fluorophore excitement against emissions from pH-dependent fluorophore excitement and against the second dye in the cell sample, the cell sample is contacted with a bioactive substance. The concentration and duration of contact will depend on the bioactive substance, the nature of the cell sample, and the experimental parameters understood by those of skill of the art of cell sample analysis or as directed by physicians, pathologists, or medical researchers.

The effect of the bioactive substance upon mitochondrial metabolism and drug efflux capacity may be measured against a control using a neutral vehicle or no substance. Methods for establishing an experimental group and control group are known in the art, and may vary depending on the cell sample and bioactive substance to be tested.

It is also possible to contact the bioactive substance with the cell sample at an earlier stage if the bioactive substance, cell sample, or experimental conditions make doing so necessary or advantageous. Specifically, it may be advantageous to contact the bioactive substance with the cell sample before the second dye if the second dye is a rhodamine, as certain rhodamine dyes may decrease membrane permeability in some cell samples.

After an initial ratiometric measurement of emissions from pH-independent fluorophore excitement against emissions from pH-dependent fluorophore excitement and against the second dye in the cell sample, subsequent ratiometric measurements are then made to determine the change in emission ratios over time in the presence of the bioactive substance. Accordingly, the effect of the bioactive substance on mitochondrial metabolism and drug efflux capacity can be determined by measuring and analyzing the change in ratiometric measurements. Even though a bioactive substance may occlude some emission or change other factors in the cell sample that determine the total fluorescent output of the dual fluorophores and second dye, the dual fluorophore dyes remain in a constant ratio of one pH-independent fluorophore to one pH-dependent fluorophore such that the ratio of their fluorescence can be used to reliably determine mitochondrial pH. The change in total fluorescence of the dual fluorophore emissions can also be used to calibrate other fluctuations, such as changes in drug efflux capacity and mitochondrial metabolism in the presence of rhodamine dyes, or in the total number of live or dead cells in the presence of or as a consequence of the previous exposure to the bioactive substance.

In some embodiments, the methods for determining the effect of a bioactive substance on mitochondrial metabolism or mitochondrial drug efflux capacity, the second fluorescent dye is a total mitochondrial potential reporting agent. In preferred embodiments, the second fluorescent dye is rhodamine 800 or rhodamine 700.

For methods for determining the effect of a bioactive substance on mitochondrial metabolism or mitochondrial drug efflux capacity, preferred embodiments will use bioactive substances which are known to not directly affect the total mitochondrial potential. As fluorescence intensities of total mitochondrial potential reporting agents such as rhodamine 800 and rhodamine 700 are used for the ratiometric analysis, bioactive substances that directly affect the total mitochondrial potential may not be suitable for such methods.

The described methods of analyzing mitochondrial function and the effect of bioactive substances on mitochondrial function may be used to identify drug candidates for improved therapies. The information provided by such analytical methods may be used in a number of ways to identify drug candidates, treatment regimens, and patient candidates for certain therapies.

Mitochondrial profiling can be particularly useful for assessing potential efficacy of Bcl2-inhibitors, such as venetoclax. In acute myeloid leukemia, venetoclax improved survival of patients for the first time in almost half a century, but the challenge remains with a large proportion of the patients being non-responders. Mitochondrial profiling as described in this invention, could reliably segment the patients who are responders and non-responders to venetoclax. Furthermore, this invention demonstrates that mitochondrial profiles of clinical leukemia patients can be screened, and that these profiles vary greatly. This information can be used to stratify leukemia patients into different clusters based on their mitochondrial profile.

Tumor cells often exhibit metabolic reprogramming involving a range of metabolic features, including aberrant mitochondrial metabolism, abnormal expression of metabolic enzymes, and increased dependence on glycolysis for ATP generation and biomolecule production. They also frequently exhibit dysregulation in other mitochondrial parameters, including deregulated mtDNA content, increased reactive oxygen species production, and defects in oxidative phosphorylation, suggesting that these alterations can be indicative of carcinogenesis. Such alterations to mitochondrial metabolism can provide tumour cells with survival advantages, contribute to the resistance to chemotherapy and can boost metastatic potential. At the same time, these alterations can render the cancer cells susceptible to metabolic therapeutics, that target specifically malignant and not the healthy cells, as healthy cells lack the metabolic alterations that are affected by these drugs. There are several types of metabolic adaptations in mitochondria of cancer cells, and an effective therapy relies on the precise diagnosis of which type is present in a particular patient.

In any of the described methods, the cell sample may comprise human cells, including human cancer cells. Cell samples may be treated with a therapeutic candidate (drug, antibody, peptide, etc.) in vitro prior to analysis. The use of the analytical methods described herein may link the mitochondrial screening to the effect of these therapeutic candidates, with the goal of building a highly accurate database (using AI/ML) of predictive mitochondrial profiles which can be used in the future to select the patients who would be responsive to a specific therapeutic candidate, or to use these mitochondrial profiles as a selection criterion to screen therapeutic candidates/lead compounds in the context of drug discovery. Alternatively, a blood, or bone marrow, or tissue homogenate sample can be taken from a patient, or animal model (e.g. mouse), treated with a therapeutic candidate. In that case, the effect of a therapeutic candidate is registered in vivo. Such effects can be survival time, degree of side effects, disease progression, quality of life, etc. These effects are used as the label vectors (Y-vectors) in AI/ML training of predictive databases, where feature vectors (X-vectors) are the quantified resulting values of the screening methodology. Another procedure involves using cells taken from an individual with the disease, or from an animal model of a disease (e.g. mouse), with the purpose of comparing it to healthy control.

As an example, a known drug may target mitochondrial function in a cancer known to have metabolic adaptations in the mitochondria of tumor cells. A first analysis of cell samples from various patients is carried out using the dual fluorophore dyes described in the present invention in combination with live cell markers, which determines that tumor cells of the cancer have more mitochondria per cell than healthy cells. Additionally, the pH-dependent fluorophore to pH-independent fluorophore ratios indicate that the mitochondrial pH of such tumor cells is higher (i.e., less acidic) than healthy cells, likely due to rapid growth and an increased rate of various mitochondrial metabolic processes, or the mitochondrial adaptations to increase tumor cell survival. A second analysis of cell samples from various patients is carried out using the dual fluorophore dyes and rhodamine 800 or rhodamine 700 described in the present invention to determine the drug efflux capacity of the mitochondria in the cell samples. This second analysis finds highly variable drug efflux capacity, such that the patients fall into two clusters: one where tumor cell mitochondria have normal drug efflux capacity, and a second where tumor cell mitochondria exhibit high drug efflux capacity by rapidly expelling the dual fluorophore dyes from the mitochondria.

As a result, a third analysis may be performed, wherein cell samples are collected from individual patients to determine whether their cancer cell mitochondria have a normal drug efflux capacity or a high drug efflux capacity. Data is collected from individual patients as they are treated for the cancer using one or various treatment therapies. Accordingly, the efficacy of treatment, presence of side effects, and survivability may be analyzed in view of the variable mitochondrial function. It may be found that patients in the cluster having tumor cells with normal drug efflux capacity respond favorably to treatment with a certain drug, while patients in the cluster having rapid drug efflux capacity see no improvement and experience significant negative side effects. As a result, future patients may be tested to determine the drug efflux capacity of their cancer cell mitochondria, and the results may better indicate or contraindicate a course of treatment.

In these ways, the described methods of measuring and analyzing mitochondrial function using the fluorescent dyes described herein can both identify potential bioactive substances and drug candidates for treating conditions that affect mitochondrial function, and can identify individual patients who may benefit from particular courses of treatment. As a result, certain therapies that had previously been avoided due to highly variable outcome results may become more widely available, as patients fitting a mitochondrial profile for positive results can be indicated for treatment, while patients fitting a mitochondrial profile for negative results can be contraindicated for treatment, increasing total efficacy of treatment.

In any of the above-described methods for measuring the effect of a bioactive substance, the bioactive substance may comprise a tumor-specific targeting drug. In any such methods, the bioactive substance may comprise a Bcl-2 inhibitor, an enzyme inducer or enzyme inhibitor for tricarboxylic acid (TCA) cycle enzymes, an electron transport chain (ETC) enzyme, a one carbon metabolism pathway enzyme, or a fatty acid β-oxidation (FAO) enzyme.

In a fifth aspect, the methods can be used to improve therapeutic efficacy in patients suffering from a disease affecting mitochondrial metabolism such as cancer, comprising:
(a) measuring a plurality of patients' cell samples using the method of claim 1 or 4;
(b) measuring the effect of a therapeutic treatment using a bioactive substance among said plurality of patients, such as efficacy, mortality, or side effects;
(c) comparing via a computer, the information on the mitochondrial function of the plurality of patients from step (a), the effect of a therapeutic treatment using a bioactive substance among said plurality of patients from step (b), wherein the computer comprises computer executable logic that provides instructions for executing the comparison;
(d) calculating the statistical correlation of the patients' mitochondrial function measurement compared to a measured effect of therapeutic treatment using a bioactive substance;
(e) measuring the mitochondrial function of an individual patient suffering from a disease affecting mitochondrial metabolism using the method of claim 1 or claim 4; and
(f) administering the bioactive substance to said individual patient if their mitochondrial function measurement correlates to the desired measured effect of therapeutic treatment using said bioactive substance.

As described above, mitochondrial function is known to affect or be affected by certain cancers, and thus therapies affecting human cancer cells are contemplated to be a preferred subject for the analytical methods described herein. Bioactive substances of particular interest are contemplated to include tumor-specific targeting drugs, specifically a Bcl-2 inhibitor, enzyme inducer or enzyme inhibitor for tricarboxylic acid (TCA) cycle enzymes, an electron transport chain (ETC) enzyme, a one carbon metabolism pathway enzyme, or a fatty acid β-oxidation (FAO) enzyme.

In one aspect of improving therapeutic efficacy in patients suffering from a disease affecting mitochondrial metabolism such as cancer, the method would comprise measuring a plurality of patients' cell samples using the methods described herein; measuring the effect of a therapeutic treatment using a bioactive substance among said plurality of patients, such as efficacy, mortality, or side effects; comparing via a computer, the information on the mitochondrial function of the plurality of patients from step (a), the effect of a therapeutic treatment using a bioactive substance among said plurality of patients from step (b), wherein the computer comprises computer executable logic that provides instructions for executing the comparison; calculating the statistical correlation of the patients' mitochondrial function measurement compared to a measured effect of therapeutic treatment using a bioactive substance; measuring the mitochondrial function of an individual patient suffering from a disease affecting mitochondrial metabolism using the methods described herein for determining or estimating mitochondrial function; and administering the bioactive substance to said individual patient if their mitochondrial function measurement correlates to the desired measured effect of therapeutic treatment using said bioactive substance.

### BRIEF DESCRIPTION OF THE DRAWINGS

FIG. 1 is an example of the dual fluorophore probes used in the described methods.

### DETAILED DESCRIPTION OF THE DRAWINGS

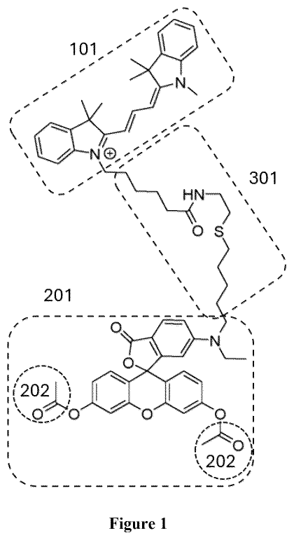

FIG. 1 illustrates a dual fluorophore used in the present invention, particularly 6AF-NDS-Cy3-DA. The pH-independent fluorophore 101 is a cyanine dye, which is represented here by cyanine-3 (Cy3). Cyanine-5 (Cy5) is also a pH-independent fluorophore contemplated by the dual fluorophore dyes of the invention. The Cy3 molecule here exhibits resonance and as such, the double bonds and iminium cation may displace internally without changing the structure. Cy5 exhibits similar mesomerism. The un-ionized pyrrolidine ring in the Cy3 of FIG. 1 is 1-methylated, but Cy3 or Cy5 containing 1-ethylated or 1-propylated pyrrolidine are expected to be functionally equivalent in terms of resonance, fluorescence, and ionicity.

The pH-dependent fluorophore 201 is 6-Aminofluorescin. In this illustration, the protecting groups 202 are acetyl groups, making this 6-Aminofluorescin diacetylated. When the molecule as shown enters the mitochondria, these protecting groups are removed, converting the fluorophore from inactive to active form. The acyl protecting groups 202 here may be replaced with propionyl, butyryl, isobutyryl, pivaloyl or benzoyl protecting groups without affecting the resonance, fluorescence, or tautomerism of the active form of the 6-aminofluorescin fluorophore, and thus are expected to be functionally equivalent. Additionally, the nitrogen of the 6-aminofluorescein in FIG. 1 is ethylated, but replacing the ethyl group with a methyl, propyl, butyl, pentyl or hexyl group would be functionally equivalent as it would not be expected to materially affect the resonance, fluorescence, or tautomerism of the 6-aminofluoride.

The linker 301 here connects the pH-independent fluorophore 101 and pH-dependent fluorophore 201 to ensure that each fluorophore is present in a 1:1 ratio with the other for ratiometric analysis. The linker 301 is a covalent chain designed to minimize any steric hindrance for the rotation of the two fluorophores 101, 201 around the linker 301 relative to each other that may adversely affect the ability of the probe to travel through the cellular plasma membrane and mitochondrial membranes. In FIG. 1, the linker 301 is —(CH2)5-(C═O)—NH—CH₂CH₂—S—(CH₂)5, but the two pentane chains therein may be replaced with ethane, propane, butane, hexane, heptane, or octane chains without materially affecting the functionality of the dual fluorophore.

1. A method of determining mitochondrial pH, the method comprising the steps of:
(a) washing a cell sample with a bicarbonate-free cell culture medium with a tightly controlled pH value;
(b) contacting the cell sample with 6AF-NDS-Cy3-DA and/or 6AF-NDS-Cy5-DA, said compound comprising a pH-independent fluorophore linked to a pH-dependent fluorophore;
(c) rinsing the cell sample with a bicarbonate-free cell culture medium with a tightly controlled pH value;
(d) measuring a ratio of fluorescence between the pH-independent fluorophore and the pH-dependent fluorophore, where the value of background fluorescence was subtracted, to increase signal-to-noise ratio; and
(e) determining the mitochondrial pH using said ratio of fluorescence.

2. The method according to claim 1, wherein the cell sample is also contacted with a dead cell marker and/or live cell marker before rinsing.

3. The method according to claim 2, wherein the dead cell marker and/or live cell marker comprises and/or is 4′,6-diamidino-2-phenylindole (DAPI) (CAS 28718-90-3), calcein violet, calcein violet AM, and/or calcein green (CAS 48504-34-1), calcein green AM.

4. A method of estimating mitochondrial drug efflux capacity, the method comprising the steps of:
(a) washing a cell sample with a bicarbonate-free cell culture medium with a tightly controlled pH value;
(b) contacting the cell sample with 6AF-NDS-Cy3-DA and/or 6AF-NDS-Cy5-DA, said compound comprising a pH-independent fluorophore linked to a pH-dependent fluorophore;
(c) rinsing the cell sample with a bicarbonate-free cell culture medium;
(d) contacting the cell sample with a second fluorescent dye;
(e) measuring a ratio of fluorescence between the pH-independent fluorophore, the pH-dependent fluorophore, and the ratio of florescence between second fluorescent dye and pH-independent fluorophore of the first compound;
(f) repeating the step (e) two or more times, and
(g) estimating mitochondrial drug efflux capacity using the change in these ratios of fluorescence over time.

5. The method according to claim 4, wherein the second fluorescent dye comprises and/or is a total mitochondrial potential reporting agent.

6. The method according to claim 5, wherein the second fluorescent dye comprises and/or is rhodamine 800 (CAS 137993-41-0) and/or rhodamine 700 (CAS 63561-42-2).

7. A method of determining the effect of a bioactive substance upon mitochondrial pH, the method comprising the steps of:
(a) washing a cell sample with a bicarbonate-free cell culture medium with a tightly controlled pH value;
(b) contacting the cell sample with 6AF-NDS-Cy3-DA and/or 6AF-NDS-Cy5-DA, said compound comprising a pH-independent fluorophore linked to a pH-dependent fluorophore;
(c) rinsing the cell sample with a bicarbonate-free cell culture medium with a tightly controlled pH value;
(d) measuring a first ratio of fluorescence between the pH-independent fluorophore and the pH-dependent fluorophore;
(e) contacting the cell sample with a bioactive substance, neutral vehicle, or leaving the sample for a period of time,
(f) measuring a second ratio of fluorescence between the pH-independent fluorophore and the pH dependent fluorophore; and
(g) determining the effect of the bioactive substance on mitochondrial pH using the first ratio of step (d) and second ratio of obtained in step (f).

8. The method according to claim 7, wherein the cell sample is also contacted with a dead cell marker and/or live cell marker before rinsing.

9. The method according to claim 8, wherein the dead cell marker and/or live cell marker comprises and/or is 4′,6-diamidino-2-phenylindole (DAPI) (CAS 28718-90-3), calcein violet, calcein violet AM, and/or calcein green (CAS 48504-34-1), calcein green AM.

10. The method of claim 7, wherein the bioactive substance comprises and/or is a tumor-specific targeting drug.

11. The method of claim 7, wherein the bioactive substance comprises and/or is a Bcl-2 inhibitor.

12. The method of claim 7, wherein the bioactive substance comprises and/or is an enzyme inducer or enzyme inhibitor for tricarboxylic acid (TCA) cycle enzymes, an electron transport chain (ETC) enzyme, a one carbon metabolism pathway enzyme, or a fatty acid β-oxidation (FAO) enzyme.

13. The method of claim 1, wherein the cell sample comprises human cells.

14. The method of claim 13, wherein the cell sample comprises human cancer cells.

15. The method of claim 4, wherein the cell sample comprises human cells.

16. The method of claim 15, wherein the cell sample comprises human cancer cells.

17. The method of claim 7, wherein the cell sample comprises human cells.

18. The method of claim 17, wherein the cell sample comprises human cancer cells.
